(19) United States
(12) Patent Application Publication
(10) Pub. No.: US 2022/0028493 A1
(43) Pub. Date: Jan. 27, 2022

(54) METHOD FOR PREDICTING ABSORBANCE CHANGE BY INTERMOLECULAR INTERACTION

(71) Applicant: INDUSTRY-ACADEMIC COOPERATION FOUNDATION, YONSEI UNIVERSITY, Seoul (KR)

(72) Inventors: Byung Chan HAN, Seoul (KR); Jin Woo OH, Busan (KR); Jong Min LEE, Gyeonggi-do (KR); Ho Je CHUN, Seoul (KR); Joon Hee KANG, Seoul (KR)

(73) Assignee: INDUSTRY-ACADEMIC COOPERATION FOUNDATION, YONSEI UNIVERSITY, Seoul (KR)

(21) Appl. No.: 17/286,106

(22) PCT Filed: Oct. 18, 2018
(86) PCT No.: PCT/KR2018/012335

§ 371 Date: Aug. 9, 2021

(30) Foreign Application Priority Data
Oct. 18, 2018 (KR) 10-2018-0124342

(51) Int. Cl. G16B 30/20; G01N 21/31; G16B 15/30

(52) U.S. Cl. CPC G16B 30/20; G16B 15/30; G01N 21/31

(57) ABSTRACT

The present disclosure relates to a method for predicting an absorbance change by intermolecular interaction, and more particularly, to a method for predicting an absorbance change by the intermolecular interaction, in which absorbance is calculated according to the type of interaction force and bond between an amino acid and a target material using first-principles calculation based on density functional theory (DFT), thereby predicting a change in optical properties when the target material is adsorbed onto 20 amino acids or a peptide composed of two or more amino acids, and screening.

### TECHNICAL FIELD

The present disclosure relates to a method for predicting an absorbance change by intermolecular interaction, and more particularly, to a method for predicting an absorbance change by intermolecular interaction, in which absorbance is calculated according to the type of interaction force and bond between an amino acid and a target material using first-principles calculation based on density functional theory (DFT), thereby predicting a change in optical properties when the target material is adsorbed onto 20 amino acids or a peptide composed of two or more amino acids, and screening.

### BACKGROUND ART

Recently, many applied research has been carried out in biosensors using a biocompatible material called ‘M13 bacteriophage’.

In general, M13 bacteriophage (hereinafter, M13 phage) is a particle having the length of 880 nm and the width of 6.6 nm, and as opposed to nanoparticles made through the general organic and inorganic synthesis, it is assembled from protein expressed through uniform genes, and all particles are exactly identical in shape.

Accordingly, there is a large advantage in the material preparation process. Additionally, it is a nanoparticle having a high surface to volume ratio, and has about 2700 pairs of proteins (pvIII protein) on the surface and 4 to 5 pairs of proteins (pill, pVI, pVII, pIX) at two ends per particle. In particular, in the case of the protein pVIII which has 2700 copies of peptides, protein molecules that form a pair with a spacing of about 3.3 nm are arranged very densely in a spiral shape. Genetic recombination in bacteriophage allows expression of a desired peptide on each corresponding surface protein, so it is easy to effectively produce functional nanoparticles of high efficiency suited for the purpose.

Additionally, as opposed to other synthesized nanoparticles, M13 phage is a material consisting of protein and a virus that commonly exists in a normal natural environment, but it is a material that infects only Escherichia coli (E.coli) having a specific strain, and so far, there have been no reported cases of mutation that harms the health of humans. In 2006, bacteriophage was approved by FDA, and is used as an additive for preventing bacterial infections in instant foods, and it is a biocompatible material that is harmless to humans as an alternative to antibiotics that can overcome the antibiotic tolerance problem. Due to these features, bacteriophage gains attention in the field of biological tissue engineering in recent years.

In particular, M13 phage gains attention as a next-generation material since it can be produced in large quantities with low labor and allows users to easily introduce desired functions, and it is possible to express a desired nucleic acid sequence at the terminal portion of M13 phage through bioengineering, thereby sensing a desired target material with higher accuracy through a specific amino acid sequence.

However, for bioengineering or designing, it is necessary to predict interaction between bacteriophage and the target material and detect a change in optical properties.

To experimentally obtain the above results, it takes a long time, and when absorbance is predicted without the entire phage synthesis, there is a limitation of the experimental measurement method in measuring a low wavelength range.

Conventionally, the amino acid sequence for phage engineering or designing is used after predicting the reactivity with the target material through functional groups of amino acids, but it is difficult to analyze the extent of actual reaction and its consequential color change.

Accordingly, to address the above-described issue, the inventors recognized the urgent need for development of a method for predicting an absorbance change by intermolecular interaction and completed the disclosure.

### DISCLOSURE

### Technical Problem

The present disclosure is directed to providing a method for predicting an absorbance change by intermolecular interaction, in which absorbance is calculated according to the type of interaction force and bond between an amino acid molecule and a target material molecule using first-principles calculation based on density functional theory (DPT), thereby predicting a change in optical properties when the target material is adsorbed onto 20 amino acids and a peptide composed of two or more amino acids, and screening.

### Technical Solution

To achieve the above-described object, the present disclosure provides a method for predicting an absorbance change by intermolecular interaction.

Hereinafter, the present disclosure will be described in more detail.

The present disclosure provides a method for predicting an absorbance change by intermolecular interaction, comprising the following steps:

(S1) predicting a structure having lowest energy of an amino acid and a target material:

(S2) analyzing an interaction force between the amino acid and the target material:

(S3) calculating S1 state of each of the amino add and the target material and S1 state of a complex compound of the amino acid and the target material: and

(S4) predicting an absorbance change using the S1 states calculated in the step S3.

In the present disclosure, the amino acid is at least one selected from the group consisting of arginine (R), histidine (H), lysine (K), aspartic acid (D), glutamic acid (E), serine (S), threonine (T), asparagine (N), glutamine (Q), cysteine (C), selenocysteine (U), glycine (G), proline (P), alanine (A), valine (V), isoleucine (I), leucine (L), methionine (M), phenylalanine (F), tyrosine (Y) or tryptophan (W).

In the present disclosure, the step S1 comprises (S1a) calculating lowest energy and frequency of the amino acid and the target material using first-principles based on density functional theory (DFT); and (S1b) identifying if the amino acid and the target material are in lowest energy and positive frequency.

In the present disclosure, the step S1a is performed again when the amino acid and the target material are not in lowest energy or positive frequency in the step S1b.

In the present disclosure, the step S2 comprises (S2a) forming the complex compound by analysis of the interaction force between the amino acid and the target material; (S2b) calculating lowest energy and frequency of the complex compound of the amino acid and the target material using first-principles based on density functional theory (DFT); and (S2c) identifying if the amino acid and the target material are in lowest energy and positive frequency.

In the present disclosure, the step S2a is performed again when the amino acid and the target material are not in lowest energy or positive frequency in the step S2c.

In the present disclosure, the step S3 comprises (S3a) calculating the S1 state of each of the amino acid and the target material and the S1 state of the complex compound of the amino acid and the target material using first-principles based on density functional theory; (S3b) analyzing molecular orbital (MO) for the S1 state of each of the amino acid and the target material and the S1 state of the complex compound; and (S3c) identifying if the S1 state of each of the amino acid and the target material and the S1 state of the complex compound is a valence excitation.

In the present disclosure, when it is not the valence excitation in the step S3c, it is a charge transfer excitation, and when it is the valence excitation in the step S3c, the valence excitation is the valence excitation in the target material or the valence excitation in the amino acid.

In the present disclosure, the step S4 includes calculating a change in the S1 state of the target material by the interaction force between the amino acid and the target material, or a change in the S1 state of the amino acid by the interaction force between the amino acid molecule and the target material molecule.

### Advantageous Effects

The method for predicting an absorbance change by intermolecular interaction according to the present disclosure calculates absorbance of an amino acid and predicts an absorbance change by interaction with a target material using relatively easy and efficient methodology to an experimentally difficult and time consuming task, thereby facilitating the synthesis of amino acid sequences of desired absorbance by presenting amino acids having a desired absorbance change in advance when a specific target material is given.

Additionally, the method for predicting an absorbance change by intermolecular interaction according to the present disclosure facilitates the synthesis of a variety of phage based sensors by synthesis of photoreactive amino acid sequences.

### DESCRIPTION OF DRAWINGS

FIG. 1 is a block diagram showing a method for predicting an absorbance change by intermolecular interaction according to the present disclosure.

FIG. 2 is a block diagram showing a process in the step S1 of a method for predicting an absorbance change by intermolecular interaction according to the present disclosure.

FIG. 3 is a block diagram showing a process in the step S2 of a method for predicting an absorbance change by intermolecular interaction according to the present disclosure.

FIG. 4 is a block diagram showing a process in the step S3 of a method for predicting an absorbance change by intermolecular interaction according to the present disclosure.

FIG. 5 is a block diagram showing a process in the step S4 of a method for predicting an absorbance change by intermolecular interaction according to the present disclosure.

FIG. 6 is a diagram showing the analysis of interaction forces between amino adds (histidine) and target materials (benzene (a), toluene (b), xylene (c), aniline (d) and toluidine (e)) in the step S2 of the present disclosure.

FIGS. 7A and 7B are diagrams showing the analysis of molecular orbital (MO) for S1 state of amino acids (histidine) and target materials (benzene (a), toluene (b), xylene (c), aniline (d) and toluidine (e)), and S1 state of complex compounds of the amino acids and the target materials in the step S3 of the present disclosure.

FIG. 8 is a diagram showing S1 states of 20 amino acids calculated in the step S3a of the present disclosure.

FIG. 9A is a diagram showing the predicted wavelength and FIG. 9B shows the actually measured wavelength as a function of transmittance (%) of amino acids (histidine) and target materials (benzene (a), toluene (b), xylene (c), aniline (d) and toluidine (e)) in the step S4 of the present disclosure.

### BEST MODE

Hereinafter, the embodiments of the present disclosure will be described in detail with reference to the accompanying drawings. The present disclosure may have a variety of modifications and may be embodied in different forms, and particular embodiments are illustrated in the drawings and will be described herein in detail. However, it should be understood that this is not intended to limit the present disclosure to the particular disclosed embodiments, and encompasses all changes, equivalents or substitutes included in the spirit and scope of the present disclosure.

The terminology used herein is for the purpose of describing particular embodiments only and is not intended to be limiting of the present disclosure. Unless the context clearly indicates otherwise, the singular forms include the plural forms as well. The term “comprises” or “includes” when used in this specification, specifies the presence of stated features, steps, operations, elements, components or groups thereof, but does not preclude the presence or addition of one or more other features, steps, operations, elements, components or groups thereof.

Unless otherwise defined, all terms including technical and scientific terms used herein have the same meaning as commonly understood by those skilled in the art. It will be understood that terms, such as those defined in commonly used dictionaries, should be interpreted as having a meaning that is consistent with their meaning in the context of the relevant art document, and will not be interpreted in an idealized or overly formal sense unless expressly so defined herein.

The present disclosure provides a method for predicting an absorbance change by intermolecular interaction, comprising the following steps.

Hereinafter, the present disclosure will be described in more detail.

Method for Predicting an Absorbance Change by Intermolecular interaction

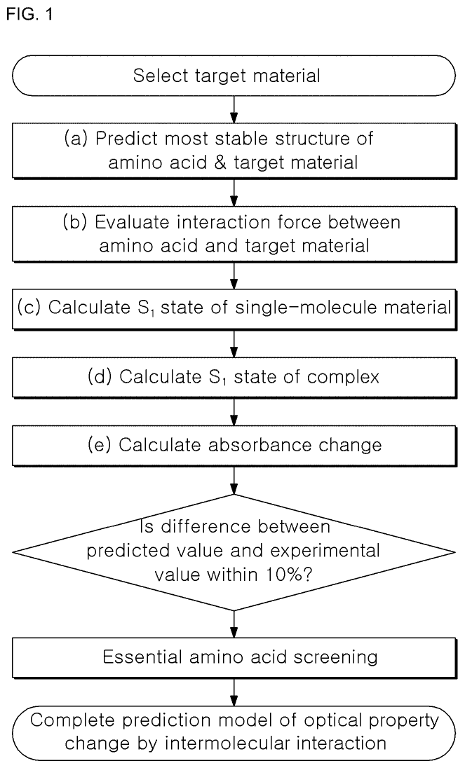

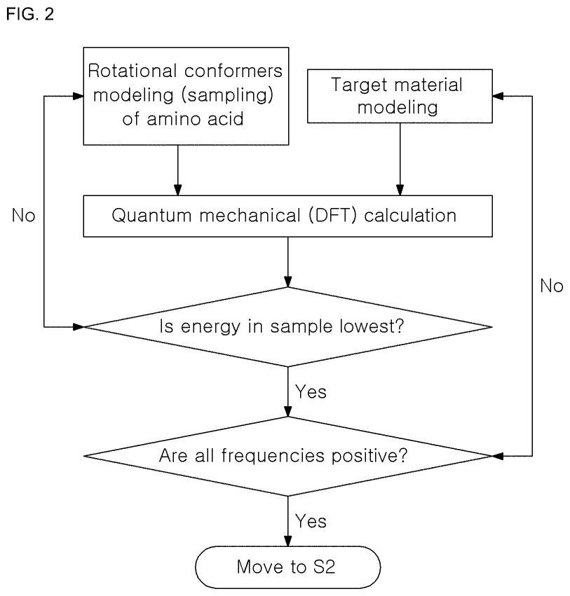

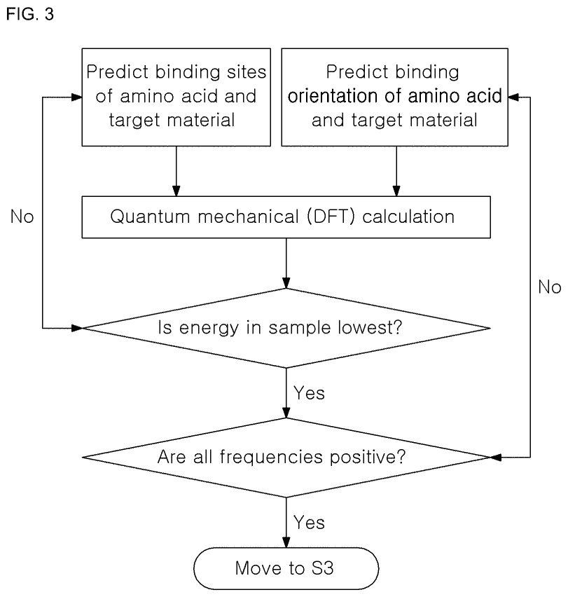

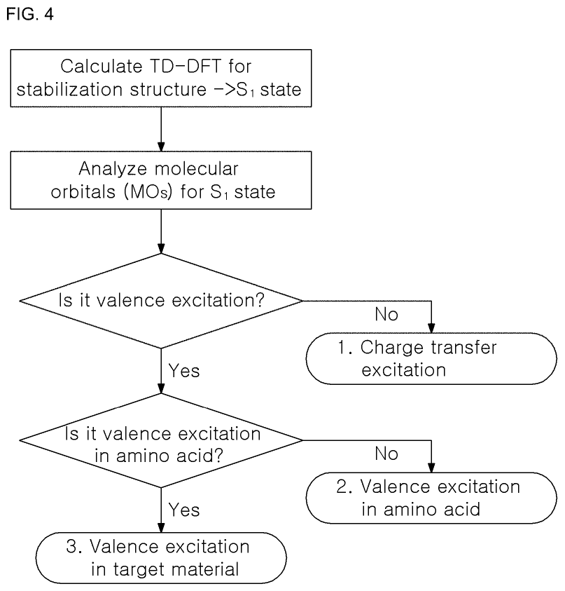

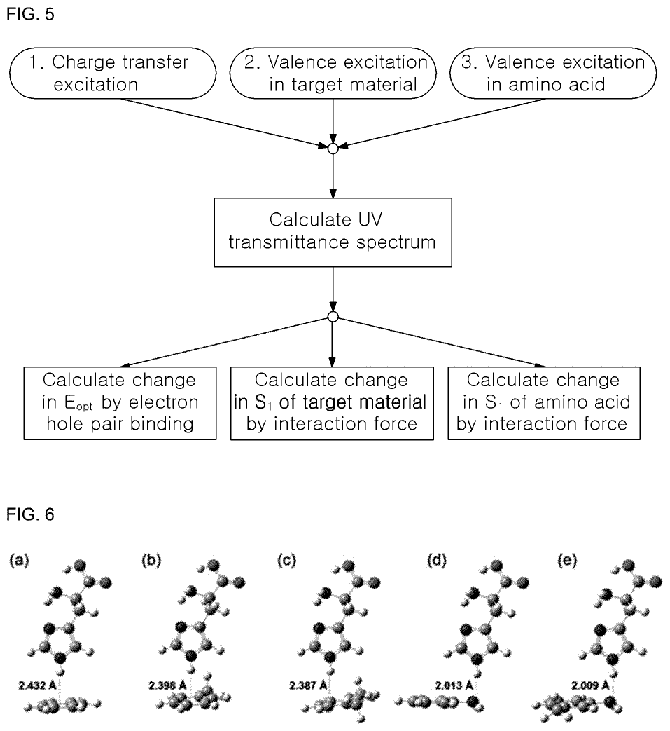

In the present disclosure, FIG. 1 is a block diagram showing a method for predicting an absorbance change by intermolecular interaction according to the present disclosure, FIG. 2 is a block diagram showing a process in the step S1 of the method for predicting an absorbance change by intermolecular interaction according to the present disclosure, FIG. 3 is a block diagram showing a process in the step S2 of the method for predicting an absorbance change by intermolecular interaction according to the present disclosure, FIG. 4 is a block diagram showing a process in the step S3 of the method for predicting an absorbance change by intermolecular interaction according to the present disclosure, and FIG. 5 is a block diagram showing a process in the step S4 of the method for predicting an absorbance change by intermolecular interaction according to the present disclosure.

More specifically, the present disclosure provides a method for predicting an absorbance change by intermolecular interaction, comprising the following steps:

(S1) predicting a structure having lowest energy of an amino acid and a target material;

(S2) analyzing an interaction force between the amino acid and the target material;

(S3) calculating S1 state of each of the amino acid and the target material and S1 state of a complex compound of the amino acid and the target material; and

(S4) predicting an absorbance change using the S1 states calculated in the step S3.

The term “structure having lowest energy” as used herein refers to the most structurally stable state of a compound or a material, considering the ring strain and the bond length.

The term “interaction force” as used herein refers to movement of electrons between compounds or materials by π-π interaction, van der Waals force and hydrogen bond, or bond (bonding force) between compounds or materials.

The term “S1 state” as used herein refers to a difference between energy potentials of an electron in ground and excited states.

In the present disclosure, the amino acid that may be used in the method for predicting an absorbance change by intermolecular interaction may be at least one selected from the group consisting of arginine (R), histidine (H), lysine (K), aspartic acid (D), glutamic acid (E), serine (S), threonine (T), asparagine (N), glutamine (Q), cysteine (C), selenocysteine (U), glycine (G), proline (P), alanine (A), valine (V), isoleucine (I), leucine (L), methionine (M), phenylalanine (F), tyrosine (Y) or tryptophan (W).

In the present disclosure, in the step Si, the structure having lowest energy of the amino acid and the target material is predicted.

More specifically, the step S1 may include (S1a) calculating lowest energy and frequency of the amino acid and the target material using first-principles based on density functional theory (DFT); and (S1b) identifying if the amino acid and the target material are in lowest energy and positive frequency.

In the present disclosure, when the amino acid and the target material are not in lowest energy or positive frequency in the step S1b, the step S1a may be performed again.

The “density functional theory (DFT)” as used herein refers to theory for quantum mechanical calculation of the electronic configuration in a material or a molecule and its energy, and the density functional theory applied to the present disclosure is a method of calculating the total energy of a system in the ground state using the density functional theory by the introduction of the electron density function, instead of the multidimensional wave function.

Conventionally, the total energy of a system was calculated by solving the Eigenfunction & Eigenvalue equation through the wave function of an electron and the Hamiltonian operator using the Schrodinger equation represented as the following [Equation 1].

$$H Ψ = E Ψ \\ [ - \frac{{h}^{2}}{2 m} {\sum }_{i = 1}^{N} {∇}_{i}^{2} + {\sum }_{i = 1}^{N} V ( {r}_{i} ) + {\sum }_{i = 1}^{N} {\sum }_{j < i} U ( {r}_{i} , {r}_{j} ) ] Ψ = E Ψ \\ [ - \frac{{h}^{2}}{2 m} {∇}^{2} + V ( R ) + {V}_{H} ( R ) + {V}_{XC} ( R ) ] {Ψ}_{i} ( R ) = {E}_{i} {Ψ}_{i} ( R ) \qquad [ Equation 1 ]$$

However, in the case of a polyelectronic system,actually, it is impossible to calculate the total energy of the system using the above [Equation 1] by interaction between electrons, and thus the Hartree-Fock (HF) method or the Post Hartree-Fock method has been used, but these methods also require a long calculation time due to a large amount of computation, and errors frequently occur in calculated values.

The present disclosure can calculate an improved result value with a lower amount of calculation using first-principles based on density functional theory.

In the present disclosure, in the step S1a, the lowest energy and frequency of the amino acid and the target material may be calculated using first-principles based on density functional theory. In this instance, the amino acid and the target material having the lowest energy refers to a compound or a material in the most structurally stable state, and the positive frequency indicates the most thermodynamically stable state. In general, the frequency having a negative value indicates a transition state exhibiting a thermodynamically unstable state.

In the present disclosure, the step S2 includes analyzing the interaction force between the amino acid and the target material.

More specifically, the step S2 may include (S2a) forming a complex compound by analysis of the interaction force between the amino acid and the target material; (S2b) calculating lowest energy and frequency of the complex compound bonded by the interaction force between the amino add and the target material using first-principles based on density functional theory; and (S2c) identifying if the amino acid and the target material are in lowest energy and positive frequency.

In the present disclosure, when the amino acid and the target material are not in lowest energy or positive frequency in the step S2c, the step S2a may be performed again.

In the present disclosure, in the step S2a, the complex compound in the most structurally or thermodynamically stable state may be formed by analysis of the interaction force between the amino acid and the target material. More specifically, the complex compound may be a complex compound of a structure having lowest energy and positive frequency by analysis of the interaction force between the amino acid and the target material.

In the present disclosure, the first-principles based on density functional theory used in the step S2b are the same as those of the step S1a.

In the present disclosure, the interaction force Eᵢₙₜ between the amino acid and the target material in the step S2a may be calculated through the following [Equation 2]

Eᵢₙₜ=E_{complex}−E_{amino acid}−E_{target} [Equation 2]

(E_{complex}: the total energy of the complex compound of the amino acid and the target material, E_{amino acid}: the total energy of the amino acid, E_{target}: the total energy of the target material)

Referring to FIG. 6, the drawing shows the analysis of the interaction force between the amino acid and the target material using histidine as the amino acid and benzene (a), toluene (b), xylene (c), aniline (d) and toluidine (e) as the target material in the step S2 of the present disclosure. The analysis shows the interaction force between histidine and benzene (a) with the binding distance of 2.432 Å, the interaction force between histidine and toluene (b) with the binding distance of 2.398 Å, the interaction force between histidine and xylene (c) with the binding distance of 2.987 Å, the interaction force between histidine and aniline (d) with the binding distance of 2.013 Å and the interaction force between histidine and toluidine (e) with the binding distance of 2.009 Å.

In the present disclosure, the first-principles based on density functional theory used in the step S2b are the same as those of the step S1a.

In the present disclosure, in the step S3, the S1 state of each of the amino acid and the target material, and the S1 state of the complex compound of the amino acid and the target material are calculated.

More specifically, the step S3 may include (S3a) calculating the S1 state of each of the amino add and the target material and the S1 state of the complex compound of the amino acid and the target material using first-principles based on density functional theory; (S3b) analyzing molecular orbital (MO) for the S1 state of each of the amino acid and the target material and the S1 state of the complex compound; and (S3c) identifying if the S1 state of each of the amino acid and the target material and the S1 state of the complex compound is a valence excitation.

In the present disclosure, when it is not a valence excitation in the step S3c, it may be a charge transfer excitation.

In the present disclosure, when it is a valence excitation in the step S3c, it may be a valence excitation in the target material or a valence excitation in the amino acid.

The term “charge transfer excitation” as used herein refers to an excited state by movement of charge between compounds or materials.

The term “valence excitation” as used herein refers to an excited state within a compound or a material, as opposed to the charge transfer excitation which is excitation by movement of charge between compounds or materials.

In the present disclosure, the S1 state of each of the amino acid and the target material, and the S1 state of the complex compound of the amino acid and the target material may be calculated through the following [Equation 3].

Eₒₚₜ=E_{fund}−E_{B} [Equation 3]

(Eₒₚₜ: energy in S1 state, E_{fund}: HOMO/LUMO gap energy, E_{B}: electron hole pair binding energy)

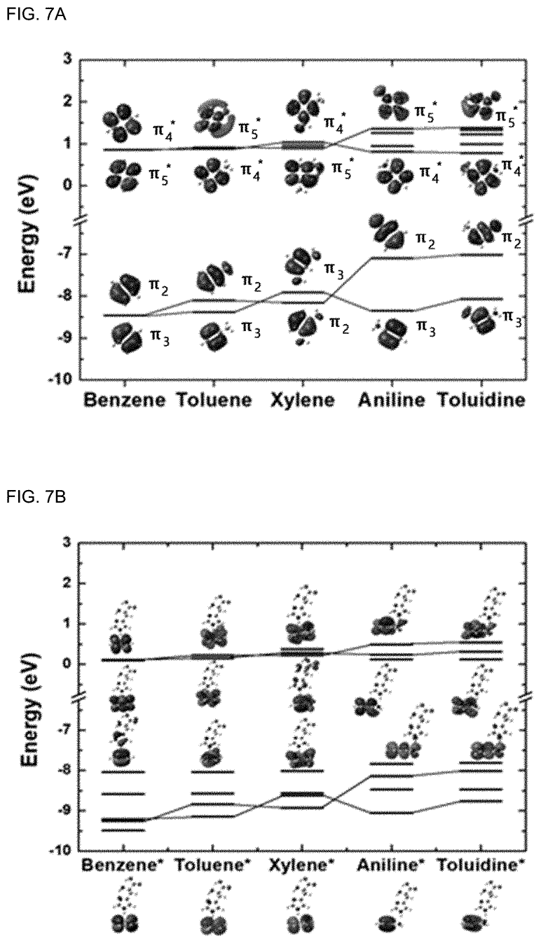

Referring to FIGS. 7A and 7B, the drawing show the analysis of molecular orbital (MO) for (FIG. 7A) the S1 state of each target material (benzene (a), toluene (b), xylene (c), aniline (d) and toluidine (e)), and (FIG. 7B) the S1 state of the complex compound of the amino acid (histidine) and the target material calculated using (S3a) the first-principles based on density functional theory of the present disclosure. It can be seen that there is a S1 state value difference between (A) the S1 state of each of the five target materials, i.e., S1 state before the interaction force occurs between the amino acid and the target material, and (B) the S1 state when the complex compound is formed by the interaction force between the amino acid (histidine) and the target material. From the above result, it is possible to predict specific optical property changes when a variety of target materials are adsorbed onto the 20 amino acids or the peptide composed of two or more amino acids.

In the present disclosure, in the step S4, the absorbance change is predicted using the S1 states calculated in S3.

More specifically, in the step S4, a change in the S1 state of the target material by the interaction force between the amino acid and the target material, or a change in the S1 state of the amino acid by the interaction force between the amino acid and the target material molecule may be calculated.

In the present disclosure, for the change in the S1 state by the interaction force between the amino acid and the target material, a S1 state difference of the complex compound of the amino acid and the target material may be calculated to calculate absorbance.

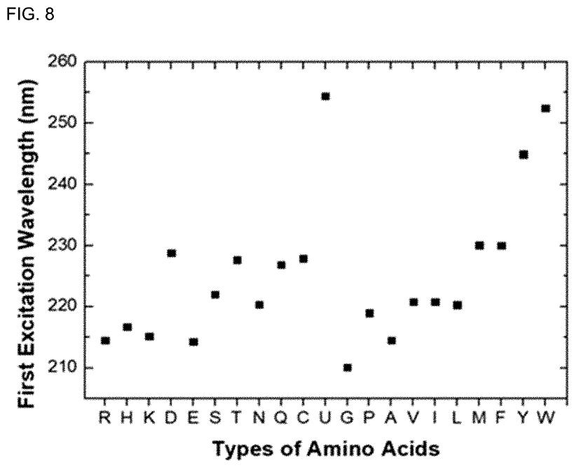

Referring to FIG. 8, the drawing shows the S1 state of the 20 amino acids calculated in the step S3a of the present disclosure.

In the present disclosure, for the change in the S1 state of the target material by the interaction force between the amino acid and the target material, when the S1 state of the target material is larger than the S1 state of the amino acid shown in FIG. 8, a difference value of the S1 state of the complex compound in the S1 state of the target material may be calculated to calculate absorbance.

In the present disclosure, for the change in the S1 state of the amino acid by the interaction force between the amino acid and the target material molecule, when the S1 state of the amino acid shown in FIG. 8 is larger than the S1 state of the target material, a difference value of the S1 state of the complex compound in the S1 state of the amino acid may be calculated to calculate absorbance.

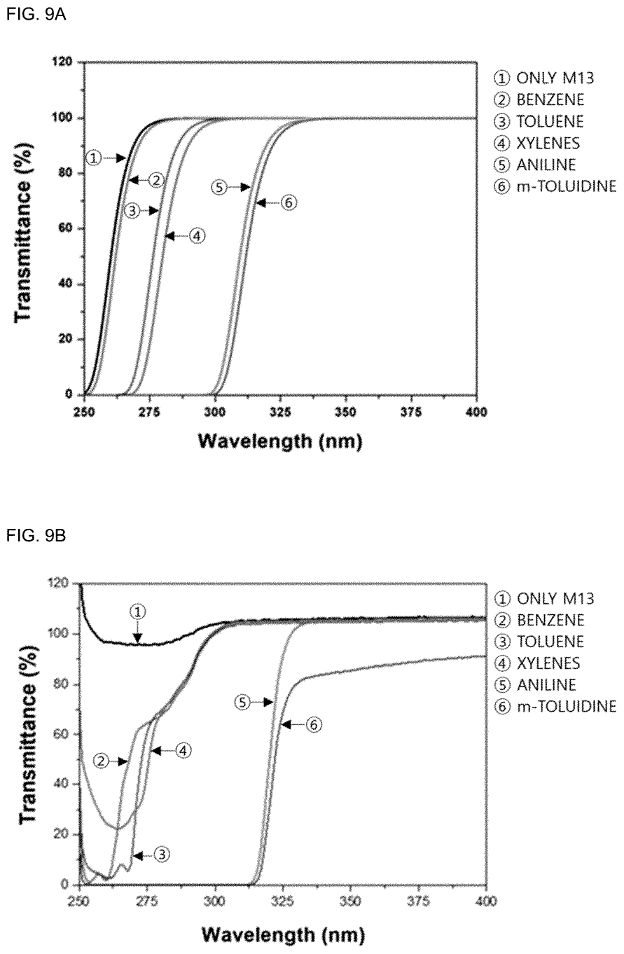

Referring to FIGS. 9A and 9B, the drawings show the predicted transmittance (%) of the amino acids (histidine) and the target materials (benzene (a), toluene (b), xylene (c), aniline (d) and toluidine (e)) in the step S4 of the present disclosure. More specifically, it can be seen that (FIG. 9A) the predicted value of transmittance (%) of the amino adds (histidine) and the target materials (benzene (a), toluene (b), xylene (c), aniline (d) and toluidine (e)) using the method of the present disclosure is significantly equal to (FIG. 9B) the wavelength range of actually measured transmittance (%) of the amino adds (histidine) and the target materials (benzene (a), toluene (b), xylene (c), aniline (d) and toluidine (e)).

From the above result, the method for predicting an absorbance change by intermolecular interaction according to the present disclosure calculates absorbance of an amino acid and predicts an absorbance change by interaction with a target material using relatively easy and efficient methodology to an experimentally difficult and time consuming task, thereby facilitating the synthesis of amino acid sequences of desired absorbance by presenting amino acids having a desired absorbance change in advance when a specific target material is given.

Additionally, the method for predicting an absorbance change by intermolecular interaction according to the present disclosure facilitates the synthesis of a variety of phage based sensors by synthesis of photoreactive amino acid sequences.

1. A method for predicting an absorbance change by intermolecular interaction, comprising:
(S1) predicting a structure having lowest energy of an amino acid and a target material;
(S2) analyzing an interaction force between the amino acid and the target material;
(S3) calculating S1 state of each of the amino acid and the target material and S1 state of a complex compound of the amino acid and the target material; and
(S4) predicting an absorbance change using the S1 states calculated in the step S3,
wherein the amino acid is at least one selected from the group consisting of arginine (R), histidine (H), lysine (K), aspartic acid (D), glutamic acid (E), serine (S), threonine (T), asparagine (N), glutamine (Q), cysteine (C), selenocysteine (U), glycine (G), proline (P), alanine (A), valine (V), isoleucine (I), leucine (L), methionine (M), phenylalanine (F), tyrosine (Y) or tryptophan (W).

2. The method for predicting an absorbance change by intermolecular interaction according to claim 1, wherein the step S1 comprises:
(S1a) calculating lowest energy and frequency of the amino acid and the target material using first-principles based on density functional theory (DFT); and
(S1b) identifying if the amino acid and the target material are in lowest energy and positive frequency,
wherein the step S1a is performed again when the amino acid and the target material are not in lowest energy or positive frequency in the step S1b.

3. The method for predicting an absorbance change by intermolecular interaction according to claim 1, wherein the step S2 comprises:
(S2a) forming the complex compound by analysis of the interaction force between the amino acid and the target material;
(S2b) calculating lowest energy and frequency of the complex compound of the amino acid and the target material using first-principles based on density functional theory (DFT); and
(S2c) identifying if the amino acid and the target material are in lowest energy and positive frequency,
wherein the step S2a is performed again when the amino acid and the target material are not in lowest energy or positive frequency in the step S2c.

4. The method for predicting an absorbance change by intermolecular interaction according to claim 1, wherein the step S3 comprises:
(S3a) calculating the S1 state of each of the amino acid and the target material and the S1 state of the complex compound of the amino acid and the target material using first-principles based on density functional theory;
(S3b) analyzing molecular orbital (MO) for the S1 state of each of the amino acid and the target material and the S1 state of the complex compound; and
(S3c) identifying if the S1 state of each of the amino acid and the target material and the S1 state of the complex compound is a valence excitation,
wherein when it is not the valence excitation in the step S3c, it is a charge transfer excitation, and when it is the valence excitation in the step S3c, the valence excitation is the valence excitation in the target material or the valence excitation in the amino acid.

5. The method for predicting an absorbance change by intermolecular interaction according to claim 1, wherein the step S4 includes calculating a change in the S1 state of the target material by the interaction force between the amino acid and the target material, or a change in the S1 state of the amino acid by the interaction force between the amino acid molecule and the target material molecule.
